(19) United States
(12) Patent Application Publication
(10) Pub. No.: US 2006/0247674 A1
(43) Pub. Date: Nov. 2, 2006

(54) String cutting balloon

(76) Inventor: Ricardo D. Roman, San Diego, CA (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 11/118,713

(22) Filed: Apr. 29, 2005

(51) Int. Cl. A61M 29/00

(52) U.S. Cl. 606/192

(57) ABSTRACT

A system for treatment of a vessel lesion comprises an expandable balloon and at least one cutting string longitudinally engaged to an exterior surface of the balloon. When the balloon is in its expanded condition, a gap is formed between the at least one cutting string and the exterior surface of the balloon.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Arterial blockages, which are also called stenosis, lesions, stenotic lesions, etc, are typically caused by the build-up of atherosclerotic plaque on the inside wall of an artery. In fact, several such stenoses may occur contiguously within a single artery. This can result in a partial, or even complete, blockage of the artery. As a result of the danger associated with such a blockage, several methods and procedures have been developed to treat stenoses. One such method is an angioplasty procedure which uses an inflatable balloon to dilate the blocked artery. A typical inflatable angioplasty device, for example, is disclosed in U.S. Pat. No. 4,896,669.

Angioplasty balloons have enjoyed widespread acceptance in the treatment of stenoses. Recent studies, however, have indicated that the efficacy of the dilation of a stenosis is enhanced by first, or simultaneously, incising the material that is creating the stenosis. Consequently, developments have been made to equip angioplasty balloons with cutting edges, or atherotomes, which are intended to incise a stenosis during the dilation procedure. For example, U.S. Pat. No. 5,196,024, U.S. Pat. No. 5,616,149 and U.S. Pat. No. 5,797,935, the entire contents of each of which are incorporated herein by reference, describe inflatable angioplasty balloons having a number of atherotomes mounted longitudinally on the surface of the balloon. Upon inflation of the balloon, the atherotomes induce a series of longitudinal cuts into the surface of the stenotic material as the balloon expands to dilate the stenosis. As a result of such cuts, the stenosis is more easily dilated, and the likelihood of damaging the artery during dilation is reduced.

Blades in many existing cutting balloon assemblies tend to be fairly rigid, particularly in the axial direction. The rigid axial structure of the blade naturally limits the blades ability to elongate with the underlying balloon material during balloon expansion at high pressure. As a result, stress between the comparatively axially rigid blade and the elongating balloon may lead to stress there between. The effect of balloon elongation is more pronounced in larger diameter balloons than in smaller diameter balloons, and is further amplified in longer balloon lengths as well.

Existing blades also tend to be fairly rigid in the transverse direction as well. This has the affect of limiting the flexibility of the balloon as it is advanced through the tortuous confines of a vessel or other body lumen.

In light of the above, it would be desirable to provide a cutting balloon that is more flexible and safer.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention is directed to several embodiments. In at least one embodiment the invention is directed to a medical balloon for use with a catheter or similar device, wherein the medical balloon is equipped with at least one cutting string or a plurality of cutting strings longitudinally engaged to the medical balloon. The balloon is configured such that, when the medical balloon is in its nominal state, a gap is formed between the cutting strings and the exterior surface of the medical balloon.

In some embodiments, the medical balloon has a varying cross-sectional shape and size along its length, such that two or more peaks are formed. Between the peaks, there is a circumferential trough which has an overall cross-sectional perimeter or shape which is less than that of the peaks. One or more cutting strings are engaged to the peaks and extend over the expanse formed by the trough, when the balloon is in its nominal condition.

In some embodiments, the peaks and/or the entire balloon may have cross-sections which may take the form of any number of shapes, such as, but not limited to, round, ovoid, rectangular, triangular, and any combination thereof.

In at least one embodiment, the circumferential trough portion of the balloon which forms the expanse between adjacent peaks may also take the form of any number of shapes, both in terms of its longitudinal cross-section and it circumferential cross-section..

In other embodiments of the invention, three or more peaks may be provided, longitudinal spaced along the balloon and interspaced with corresponding circumferential troughs. The cutting strings may be engaged to two or more peaks and may extend from only one peak to an adjacent peak or they may extend up to and including the length of the balloon.

In at least one embodiment one or more portions of the cutting string(s) that are in close proximity to the balloon surface are engaged to the balloon surface by an adhesive or other mounting material. The adhesive may be any adhesive material suitable for securing a metal, polymer or carbon based blade to the material of the balloon. Other bonding methods may be used, including, but not limited to, heat bonding, laser welding, RF bonding and hot jaw.

As indicated above, a balloon may be equipped with any number of cutting strings, as desired. Multiple cutting strings may be uniformly or irregularly spaced apart.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a perspective view of an embodiment of the invention wherein a balloon is shown with multiple cutting strings.

FIG. 2 is a perspective view of an embodiment of the invention wherein a balloon is shown with multiple cutting strings.

FIG. 3 is a perspective view of an embodiment of the invention wherein a balloon is shown with multiple cutting strings.

FIG. 4 is a perspective view of an embodiment of the invention wherein a balloon is shown with multiple cutting strings.

FIG. 5 is a perspective view of an embodiment of the invention wherein a balloon is shown with multiple cutting strings.

FIGS. 6a-6c show representative configurations of embodiments of the invention.

FIG. 7 is a perspective view of an embodiment of the invention wherein a collapsed balloon is shown with multiple cutting strings.

FIG. 8 is a cross-sectional view of the embodiment shown in FIG. 7 along lines 8-8.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

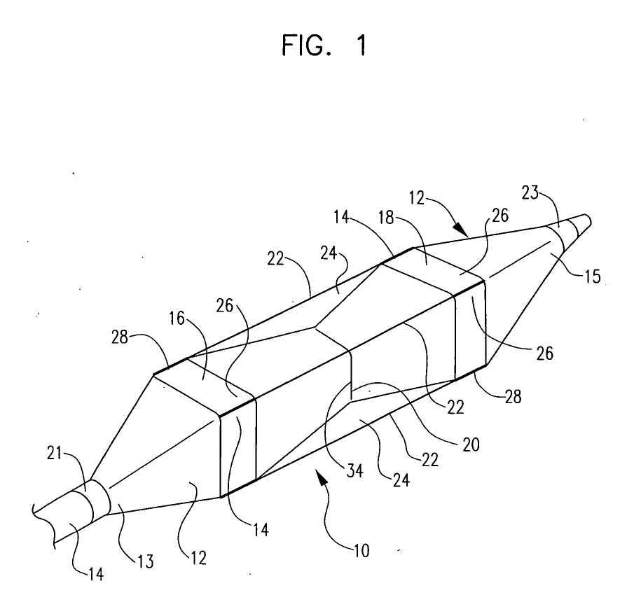

As indicated above, the present invention is embodied in a variety of forms. In at least one embodiment, an example of which is depicted in FIG. 1, the invention is directed to a cutting balloon catheter, the distal end of which is generally shown at 10. In this particular embodiment, a balloon 12 in mounted on distal end of a catheter shaft 14. The balloon 12 has a proximal end cone 13 and a distal end cone 15, proximal 21 and distal 23 waists, and, when in its nominal state, a varying radial cross-sectional shape along its length, forming at least two peaks 16, 18. A peak is a portion of the balloon which has a greater cross-sectional shape, when the balloon is in its nominal state, than portions adjacently proximal and distal. The radial cross-sectional shapes of the peaks 16, 18, are greater than that of a balloon portion 20, positioned longitudinally there between.

In the particular embodiment shown in FIG. 1, the radial cross-sectional shapes of the peaks 16, 18, and the balloon portion 20 are rectangular in shape. However, it should be understood that the invention contemplates that the radial cross-sectional shape of the balloon may take the form of other shapes. In some instances, a balloon which takes the form of other shapes may be constructed such that, when fully pressurized, it takes on a round shape. The shapes illustrated in the figures demonstrate the shape of balloon when it is in its un-expanded or natural shape, which is their nominal or as-molded shape, which is the shape the balloon is molded to. When the balloon is expanded under pressurization, it takes on a round or rounder cross-sectional shape to form its expanded state.

The embodiment shown in FIG. 1 further includes a plurality of cutting strings 22. These cutting strings 22 are engaged with the peaks 16, 18, and are axially oriented relative to the balloon 12. As shown, when the balloon 12 is in its nominal state, the cutting strings 22 are taught and straight and may be used to cut plaque or calcified lesions. Gaps 24 are formed between the cutting strings 22 and the balloon 12, in this particular case, the balloon portion 20.

The cutting strings 22 are engaged with the peaks 16, 18, of the balloon 12 in any of a variety of ways such as by mechanical engagement, direct welding, through the use of an adhesive, etc. In the embodiment shown, an adhesive material is used to engage the cutting strings 22 to the cross-sectional top surfaces 26 of the peaks 16, 18, of the balloon 10. Any suitable adhesive may be utilized as the adhesive material. For example, adhesives such as, but not limited to, epoxy, cyanoacrolyte, urethane and UV adhesives, etc. and/or combinations of such materials may by utilized as the adhesive material.

In the particular embodiment shown in FIG. 1, as mentioned above, the peaks 16, 18, have a rectangular radial cross-sectional shape. As shown, the cutting strings 22 are engaged with the cross-sectional corners 28 of the peaks 16, 18. As shown, the comers 28 may be truncated so as to provide a flat surface to better receive the cutting strings 22. In the embodiment shown, there are four cutting strings 22. It should be understood that the cutting strings 22 may extend and be connected to or near the end cones 13, 15. The ends of the cutting strings 22 may also be connected to the waists 21, 23, of the balloon 12.

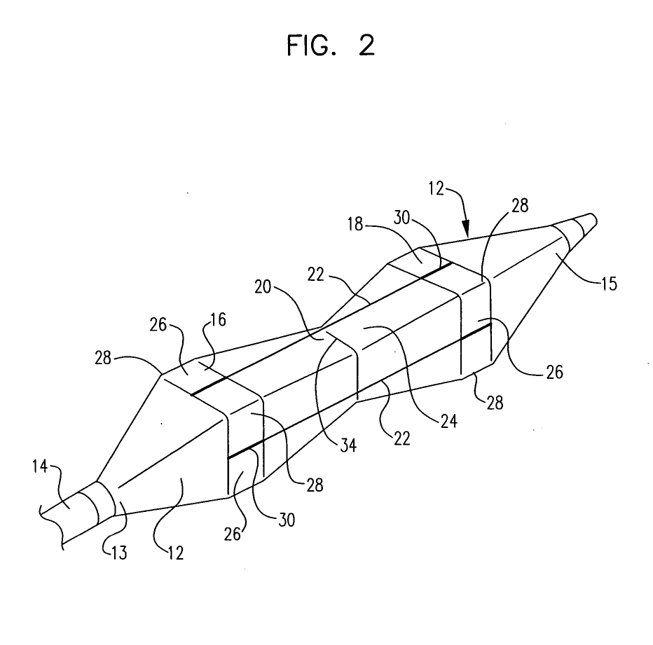

FIG. 2 illustrates a further embodiment. In this particular embodiment, the cutting strings are engaged with the top surfaces 26 of the peaks 16, 18, at points 30 between the cross-sectional comers 28 of the peaks 16, 18. Similarly, gaps 24 are formed between the cutting strings 22 and the circumferential trough 20. In the embodiment shown, there are four cutting strings 22.

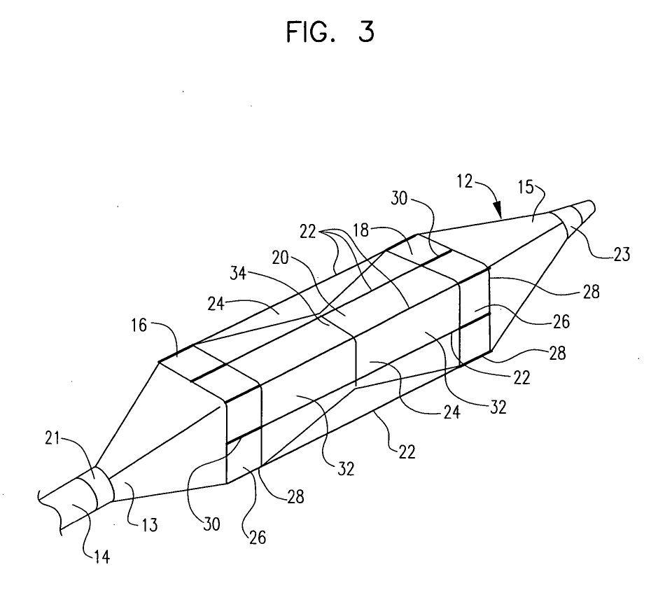

FIG. 3 illustrates a further embodiment of the invention. In this particular embodiment, the cutting strings are engaged with the top surfaces 26 of the peaks 16, 18, at points 30 between the cross-sectional comers 28 of the peaks 16, 18, and to the cross-sectional comers 28 of peaks 16, 18. Similarly, gaps 24 are formed between the cutting strings 22 and the circumferential trough 20. In the embodiment shown, there are eight cutting strings 22.

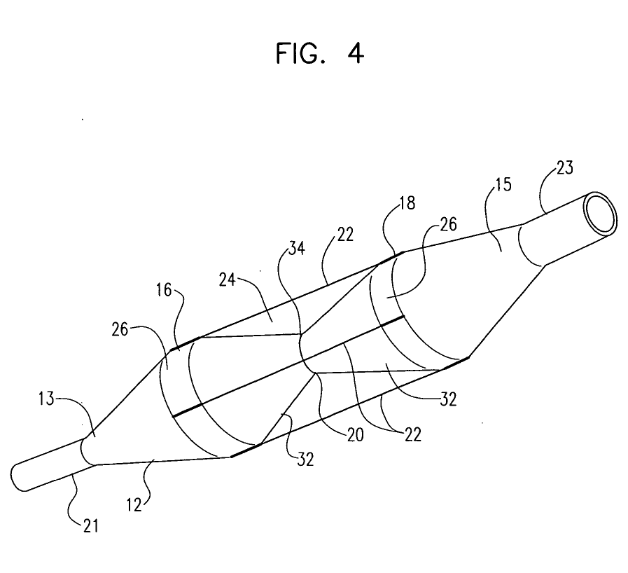

FIG. 4 illustrates a further embodiment of the invention. In this particular invention the radial cross-sectional shapes of the peaks 16, 18, and the balloon portion 20 are circular or round in shape. The cutting strings 22 are similarly engaged to the top surfaces 26 of the peaks 16, 18. Although the cutting strings 22 are shown to be parallel with the axis of the catheter shaft 14 in this and other embodiments, it should be understood that they may be positioned on a bias relative to the axis of the catheter shaft 14. It should also be understood that the radial circumference, or cross-sectional shape, of the peaks 16, 18, and circumferential trough 20 may take other shapes, such as, but not limited to, round, ovoid, rectangular, triangular, and any combination thereof.

The circumferential troughs 20 shown in FIGS. 1-4 have gradually decreasing radial cross-sectional shapes from respective peaks 16, 18, to an intermediate point 34 to form sloping balloon walls 32. However, it should be understood that the circumferential troughs 20 may take other forms. The form to be used provides an expanse between adjacent peaks, in this case peaks 16 and 18, so as to create the gap 24 between the balloon 12 and the cutting wires 22.

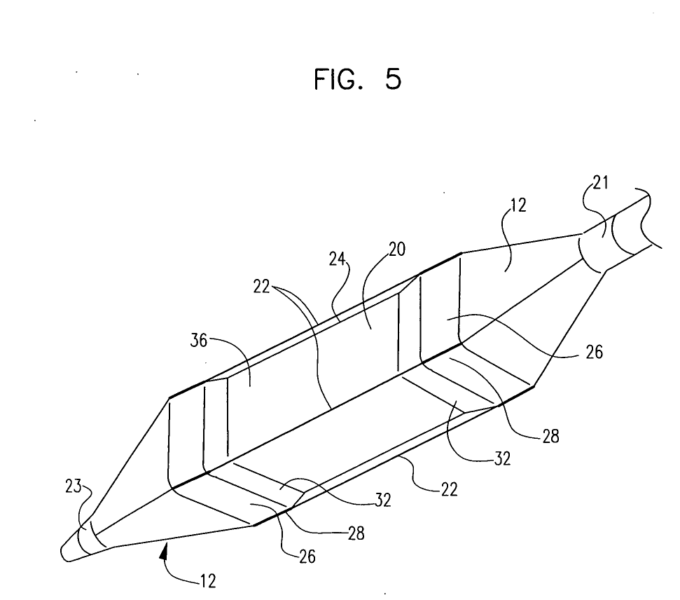

FIG. 5 illustrates an alternative form of the circumferential trough 20. In this embodiment the trough 20 provides an expanse between peaks 16 and 18 by including longitudinally shorter sloping balloon walls 32 and an elongated portion 36 of the balloon 12, which has a relatively constant radial cross-sectional shape, there between. The gap 24 is similarly formed between the balloon 12 and the cutting strings 22.

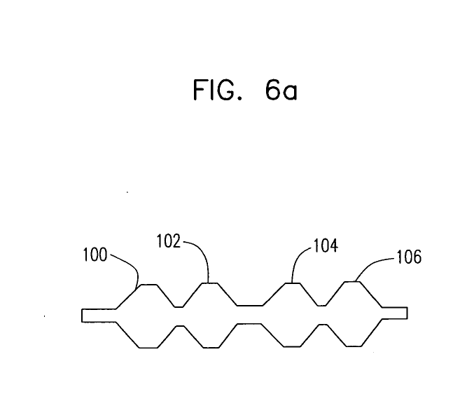

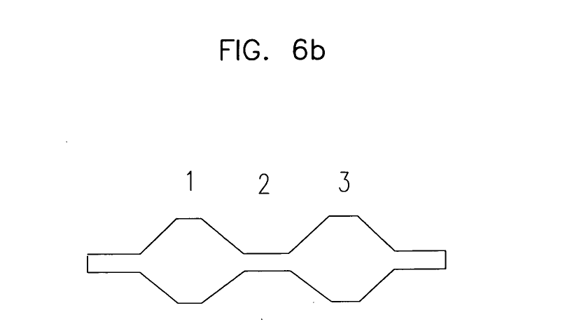

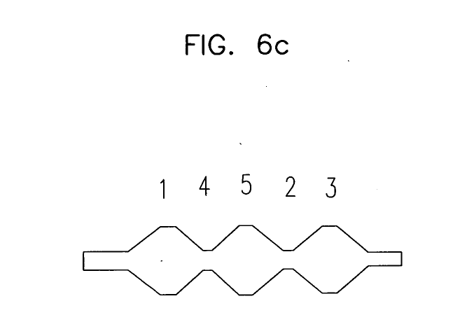

As mentioned above, the shape of the circumferential trough 20 may vary in order to provide an expanse between the adjacent peaks. The number of longitudinal peaks and corresponding circumferential trough(s) may also vary. FIGS. 6a-6c illustrates further balloon configurations. In these particular situations, the cuttings strings 22 may be connected to each peak they cross or just the most proximal and distal peaks. It should also be understood that the cutting strings 22 may be staggered on a balloon having three or more peaks, such as the one shown in FIG. 6a, which is a four peak 100, 102, 104, 106, balloon. For example, a cutting string may extend from the first peak 100 to the second peak 102, another cutting string from the second peak 102 to the third peak 104 and another from the third peak 104 to the fourth peak 106. The cutting strings may also be staggered relative to one another around the balloon.

It should be understood that the present invention contemplates varying numbers of cutting strings 22 engaged at various positions on the balloon peaks 16, 18.

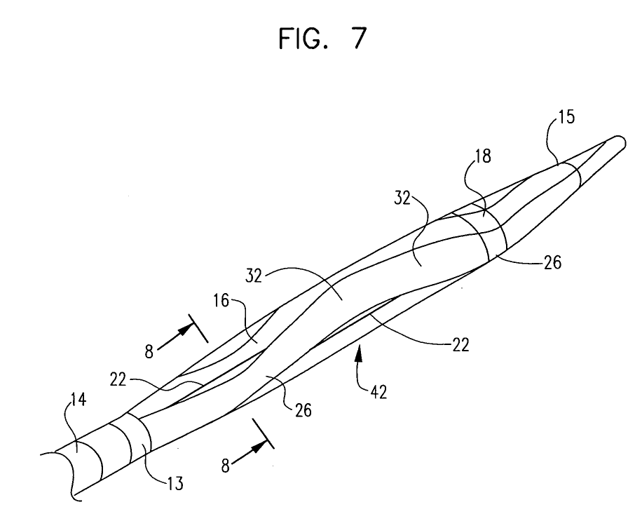

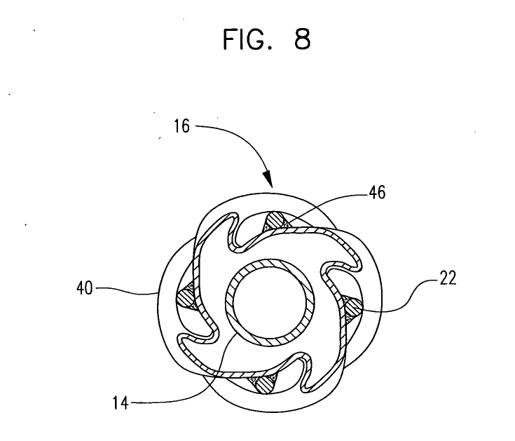

FIG. 7 is a illustration of the balloon of FIGS. 1, 2, 4 and 5 in its collapsed and folded configuration. FIG. 8 is a cross-section of the catheter of FIG. 7 along lines 8-8. As can be seen, the balloon is configured such that, when the balloon is collapsed, the portions of the peaks 40 of the balloon 12, which are circumferentially between adjacent cutting strings 22, fold over the cutting strings 22, which may be held down with adhesive 46, to cover them. Due to the decrease in the radial cross-sectional shape toward the middle of the balloon 42, portions of the cutting strings are exposed. The folding may be induced by known methods, such as, but not limited to, memory inducement using ironing techniques.

The cutting strings 22 are made of suitable material for cutting the particular obstruction within the target vessel. Such material includes, but is not limited to, one or more metals, polymers, suture strings or threads, combinations of one or more metals and/or polymers, and/or other desired material(s). If a soft material is used, the cross-sectional shape is of little matter, however, if a harder material, the cross-sectional shape may be, but not limited to, round, ovoid, ellipsoid, square, triangular, or any other geometric shape that may be desired. The cutting string 22, regardless of its cross-sectional shape or shapes may be constructed by any of a variety of manufacturing methods. For example, the string 22 may be a wire constructed of metallic or other material wire stock. Other manufacturing techniques include photo-etching, laser cutting, waterjet cutting, or flat stock stamping of a desired blade material to form the cutting string 22.

In some embodiments, the cutting string 22 may include one or more areas, coatings, materials, etc. that is (are) detectable by imaging modalities such as X-Ray, MRI or ultrasound. In some embodiments at least a portion of the cutting string is at least partially radiopaque.

While the embodiments shown in the figures have cutting strings 22 arranged in a symmetrical fashion about the balloon 12, such symmetry need not be the case in all embodiments. In some embodiments, particularly the balloons that incorporate three or more peaks, the cutting strings may be of different or equal lengths; varyingly spaced apart, whether randomly or in accordance with a pattern; or otherwise arranged or positioned about the balloon in accordance with need, desire and/or performance.

In at least one embodiment, the cutting strings 22, and/or the balloon 12 may be configured to deliver one or more therapeutic agents to the lesion site. A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The balloon 12 may be made of any suitable balloon material including compliant and non-compliant materials and combinations thereof. Some examples of suitable materials for constructing the balloon 10 include but are not limited to: low pressure, relatively soft or flexible polymeric materials, such as thermoplastic polymers, thermoplastic elastomers, polyethylene (high density, low density, intermediate density, linear low density), various co-polymers and blends of polyethylene, ionomers, polyesters, polyurethanes, polycarbonates, polyamides, poly-vinyl chloride, acrylonitrile-butadiene-styrene copolymers, polyether-polyester copolymers, and polyetherpolyamide copolymers; copolymer polyolefin material available from E.I. DuPont de Nemours and Co. (Wilmington, Del.), under the trade name Surlyn™; ionomer and a polyether block amide available under the trade name PEBAX™; high pressure polymeric materials, such as thermoplastic polymers and thermoset polymeric materials, poly(ethylene terephthalate) (commonly referred to as PET), polyimide, thermoplastic polyamide, polyamides, polyesters, polycarbonates, polyphenylene sulfides, polypropylene and rigid polyurethane; one or more liquid crystal polymers; and combinations of one or more of any of the above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A system for treatment of a vessel lesion comprising:
a catheter shaft, the catheter shaft having a proximal end portion and a distal end portion;
a medical balloon coaxially mounted on the distal end portion of the catheter shaft, the medical balloon having a proximal end, a distal end and a body portion between the proximal and distal ends of the medical balloon, said body portion having an exterior surface, wherein the medical balloon has an expanded condition, a nominal condition and a contracted condition, the medical balloon being expandable from its contracted condition to its nominal condition and from its nominal condition to its expanded condition, the body portion of the medical balloon, when in its nominal condition, having varying radial cross-sectional size along its length;
a first cutting string, the first cutting string having a first end and a second end and a length defined thereby, wherein the first end and the second end of the first cutting string are engaged with the exterior surface of the body portion of the medical balloon and wherein, when the medical balloon is in its nominal condition, a gap is formed between the first cutting string and the exterior surface of the medical balloon between the first and second ends of the first cutting string.

2. The system of claim 1, the medical balloon, when in its nominal condition, having first, second and third radial cross-sectional portions, each radial cross-sectional portion having a radial cross-sectional size, the radial cross-sectional portions being longitudinally spaced along the length of the medical balloon, wherein the second radial cross-sectional portion is between the first and third radial cross-sectional portions and wherein the first and third radial cross-sectional portions have greater radial cross-sectional sizes than that of the second radial cross-sectional portion and wherein the gap is formed between the first cutting string and the second radial cross-sectional portion of the medical balloon.

3. The system of claim 2, wherein the first cutting string is engaged to the first and third radial cross-sectional portions.

4. The system of claim 2, further comprising at least one of the following:
a second cutting string, the second cutting string having a first end and a second end and a length defined thereby, wherein the first end and the second end of the second cutting string are engaged with the exterior surface of the body portion of the medical balloon and wherein, when the medical balloon is in its nominal condition, a gap is formed between the second cutting string and the second radial cross-sectional portion of the medical balloon;
second and third cutting strings, the second and third cutting strings each having a first end and a second end and a length defined thereby, wherein the first ends and the second ends of the second and third cutting strings are engaged with the exterior surface of the body portion of the medical balloon and wherein, when the medical balloon is in its nominal condition, a gap is formed between the second and third cutting strings and the second radial cross-sectional portion of the medical balloon; and
second, third and fourth cutting strings, the second, third and fourth cutting strings each having a first end and a second end and a length defined thereby, wherein the first ends and the second ends of the second, third and fourth cutting strings are engaged with the exterior surface of the body portion of the medical balloon and wherein, when the medical balloon is in its nominal condition, a gap is formed between the second, third and fourth cutting strings and the second radial cross-sectional portion of the medical balloon.

5. The system of claim 4, wherein, when the medical balloon is in its nominal condition, the cutting strings are parallel.

6. The system of claim 4, wherein the cutting strings are engaged to the first and third radial cross-sectional portions.

7. The system of claim 4, wherein cutting strings are adhesively engaged to the external surface of the medical balloon.

8. The system of claim 4, wherein, when the medical balloon is in its nominal condition, the cutting strings are substantially linear.

9. The system of claim 4, wherein, when the medical balloon is in its nominal condition, the cross-sectional shape of the first and third radial cross-sectional portions is selected from at least one member of the group consisting of, substantially round, substantially triangular, substantially ovoid, substantially rectangular, and any combination thereof.

10. The system of claim 9, wherein, when the medical balloon is in its nominal condition, the cross-sectional shape of the first and third radial cross-sectional portions is substantially rectangular.

11. The system of claim 10, wherein the cutting strings are engaged with the corners of the first and third radial cross-sectional portions.

12. The system of claim 10, wherein the cutting strings are engaged with the sides of the first and third radial cross-sectional portions.

13. The system of claim 10, the system comprising up to 8 cutting strings, wherein the cutting strings are engaged with the corners and/or the sides of the first and third radial cross-sectional portions.

14. The system of claim 9, wherein, when the medical balloon is in its nominal condition, the cross-sectional shape of the first and third radial cross-sectional portions is substantially rectangular.

15. The system of claim 4, wherein, when the medical balloon is in its nominal condition, the radial cross-sectional size of the second radial cross-sectional portion longitudinally decreases from the first radial cross-sectional portion and from the third radial cross-sectional portion to a common point.

16. The system of claim 4, wherein, when the medical balloon is in its nominal condition, the radial cross-sectional size of the second radial cross-sectional portion longitudinally decreases from the first radial cross-sectional portion and from the third radial cross-sectional portion to a longitudinal central portion of the second radial cross-sectional portion, wherein the longitudinal central portion has a substantially constant radial cross-sectional size along its length.

17. The system of claim 4, wherein, when the medical balloon is in its nominal condition, the system further comprises fourth and fifth radial cross-sectional portions, each radial cross-sectional portion having a radial cross-sectional size, the radial cross-sectional portions being longitudinally spaced along the length of the medical balloon, wherein the fourth radial cross-sectional portion is between the first and second radial cross-sectional portions and the fifth radial cross-sectional portion is between the fourth and second radial cross-sectional portions and wherein the first, third and fifth radial cross-sectional portions have greater radial cross-sectional sizes than that of the second and fourth radial cross-sectional portions.

18. The system of claim 17, wherein, when the medical balloon is in its nominal condition, the system further comprises sixth and seventh radial cross-sectional portions, each radial cross-sectional portion having a radial cross-sectional size, the radial cross-sectional portions being longitudinally spaced along the length of the medical balloon, wherein the sixth radial cross-sectional portion is between the first and fourth radial cross-sectional portions and the seventh radial cross-sectional portion is between the sixth and fourth radial cross-sectional portions and wherein the first, third, fifth and seventh radial cross-sectional portions have greater radial cross-sectional sizes than that of the second, fourth and sixth radial cross-sectional portions.

19. The system of claim 2, wherein 1 to about 8 cutting strings are engaged to the exterior surface of the balloon.

20. The system of claim 19, when the medical balloon is in its nominal condition, the cutting strings are distributed symmetrically about the exterior surface of the balloon.

21. The system of claim 17, wherein at least a portion of the cutting string is radiopaque.

22. The system of claim 2, wherein the system is configured to deliver at least one therapeutic agent.

23. A system for treatment of a vessel lesion comprising:
a catheter shaft, the catheter shaft having a proximal end portion and a distal end portion;
a medical balloon coaxially mounted on the distal end portion of the catheter shaft, the medical balloon having a proximal end, a distal end and a body portion between the proximal and distal ends of the medical balloon, said body portion having an exterior surface, wherein the medical balloon has an expanded condition, a nominal condition and a contracted condition, the medical balloon being expandable from its contracted condition to its nominal condition and from its nominal condition to its expanded condition;
at least one cutting string, the at least one cutting string having a first end and a second end and a length defined thereby, wherein the first end and the second end of the at least one cutting string are engaged with the exterior surface of the body portion of the medical balloon and wherein, when the medical balloon is in its nominal condition, the at least one cutting string is straight and a gap is formed between the at least one cutting string and the exterior surface of the medical balloon.
